(19) United States
(12) Patent Application Publication
(10) Pub. No.: US 2025/0049689 A1
(43) Pub. Date: Feb. 13, 2025

(54) COMBINATION OF A HIGH MOLECULAR WEIGHT CROSS-LINKED HYALURONIC ACID AND A LOW MOLECULAR WEIGHT NON-CROSS-LINKED HYALURONIC ACID

(71) Applicant: L V M H RECHERCHE, Saint-Jean De Braye (FR)

(72) Inventors: Sabrina MANIGUET, Saint Jean De Braye (FR); Valérie BOUCHARD DE LA POTERIE, Saint-Jean De Braye (FR)

(73) Assignee: L V M H RECHERCHE, Saint-Jean De Braye (FR)

(21) Appl. No.: 18/717,596

(22) PCT Filed: Dec. 8, 2022
(86) PCT No.: PCT/FR2022/052285

§ 371 Date: Jun. 7, 2024

(30) Foreign Application Priority Data
Dec. 10, 2021 (FR) FR2113302

(51) Int. Cl. A61K 8/73; A61Q 19/00; A61Q 19/08

(52) U.S. Cl. CPC A61K 8/735; A61Q 19/001; A61Q 19/08

(57) ABSTRACT

The present invention relates to a cosmetic composition comprising, in a physiologically acceptable medium, the combination of at least one high molecular weight cross-linked hyaluronic acid and a low molecular weight non-cross-linked hyaluronic acid as defined in the invention.

### TECHNICAL FIELD OF THE INVENTION

The present invention concerns the field of cosmetics. It especially concerns the particular combination of a high molecular weight cross-linked hyaluronic acid and a low molecular weight non-cross-linked hyaluronic acid, intended to provide an immediate smoothing effect and/or an increase in the density and/or volume of keratin materials, especially of the skin and/or lips.

### STATE OF THE ART

Hyaluronic acid is one of the essential constituents of the extracellular matrix. On the skin, it is synthesized by fibroblasts from which it is secreted into the extracellular space. Hyaluronic acid is especially involved in the structure and organization of the dermis, helping to ensure its firmness and elasticity. In this organization, tight junctions play a key role in the regulation of water balance and permeability of the epidermis (Furuse et al., 2002. J Cell Biol. 156, 1099-1111, Morita et al., 2011. J Derm Sci. 31, 81-89). Likewise, fibrillin-1, a 340 kDa glycoprotein, is an essential component of the elastic network of the dermis. In addition, hyaluronic acid has a strong water retention capacity. It therefore plays a vital role in regulating skin hydration.

For many years, it has been possible to produce hyaluronic acid industrially, especially by bacterial fermentation, and the cosmetic industry commonly uses this molecule for its anti-aging and moisturizing properties.

Structurally, hyaluronic acid is a polymer formed by the repetition of disaccharide monomers, themselves made up of glucuronic acid and N-acetylglucosamine linked together by alternating B-1,4 and B-1,3 glycosidic bonds. Like any polymer, hyaluronic acid is a macromolecule whose molar mass can vary, depending on the number of repetitions, from a few thousand Daltons to several million (approximately 5000 to 20,000,000 Da).

To obtain an immediate smoothing effect, which is also an anti-aging factor, it is common to add fillers to cosmetic formulations to blur the visual and tactile irregularities of the skin and thus reduce the signs of aging, including fine lines and wrinkles, through an optical effect. However, it is known that fillers negatively impact the moisturizing effect provided by hyaluronic acid.

There is therefore a need to provide new moisturizing cosmetic compositions which also provide an immediate smoothing effect for the skin and/or lips.

The Applicant has precisely developed new cosmetic compositions to meet this need. Indeed, surprisingly, the Applicant was able to demonstrate that the particular combination of at least one high molecular weight cross-linked hyaluronic acid and at least one low molecular weight non-cross-linked hyaluronic acid makes it possible to significantly increase hydration and the immediate smoothing effect for keratin materials. The Applicant was also able to show that this combination makes it possible to obtain a smoothing and plumping effect on the lips and the area surrounding them;

an improvement in the smooth, soft and plump appearance of the lips was especially noted. However, the combination of at least one high molecular weight non-cross-linked hyaluronic acid and at least one low molecular weight non-cross-linked hyaluronic acid does not make it possible to obtain the desired performance, highlighting the essential role of cross-linking of the high molecular weight hyaluronic acid.

This particular combination of hyaluronic acids according to the invention is therefore particularly advantageous for providing both a moisturizing effect and an immediate smoothing effect for the skin and/or lips and thus preventing and/or reducing the signs of skin aging and/or or conferring a volumizing, lifting and smoothing effect on the lips as well as preventing and/or reducing the appearance of wrinkles.

### SUMMARY OF THE INVENTION

A first aspect of the invention relates to a cosmetic composition comprising, in a physiologically acceptable medium, the combination of at least one high molecular weight cross-linked hyaluronic acid having a molecular weight greater than or equal to 2000 kDa, or one of its salts, and a low molecular weight non-cross-linked hyaluronic acid having a molecular weight less than or equal to 300 kDa, or one of its salts.

Preferably, said cosmetic composition is characterized in that said high molecular weight cross-linked hyaluronic acid has a molecular weight greater than or equal to 3000 kDa.

Preferably, said cosmetic composition is characterized in that said low molecular weight non-cross-linked hyaluronic acid has a mean molecular weight less than or equal to 200 kDa.

Preferably, said cosmetic composition is characterized in that said high molecular weight cross-linked hyaluronic acid has the INCI name: SODIUM HYALURONATE CROSSPOLYMER.

Preferably, said cosmetic composition is characterized in that said low molecular weight non-cross-linked hyaluronic acid is chosen from products of INCI name:
SODIUM HYALURONATE, and
HYALURONIC ACID.

Preferably, said cosmetic composition is characterized in that said high molecular weight cross-linked hyaluronic acid has a cross-linking of 0.001% to 5%, preferably 0.1% to 1%, preferably 0.1% to 0.6%.

Preferably, said cosmetic composition is characterized in that said high molecular weight cross-linked hyaluronic acid and said low molecular weight non-cross-linked hyaluronic acid are each present in the composition in a content ranging from 0.001% to 10%, preferably 0.005% to 5%, more preferably from 0.01% to 1% by weight of raw material relative to the total weight of the composition.

Preferably, said cosmetic composition is characterized in that it further comprises at least one cosmetic adjuvant chosen from the group consisting of antioxidant agents, emollient agents, moisturizing agents, anti-aging agents, fragrances, and mixtures thereof.

Preferably, said cosmetic composition is characterized in that it comprises less than 5% filler, in particular less than 2% filler, preferably less than 1% filler or even is devoid of filler.

Preferably, said cosmetic composition is characterized in that it is in the form of a topical composition for the skin and/or lips, in particular in the form of a cream, oil-in-water or water-in-oil emulsion or multiple emulsion, suspension, gel, milk, lotion, serum, mask or lip balm.

According to another aspect, the present invention relates to a cosmetic process for caring for keratin materials, in particular the skin and/or lips, comprising the topical application to said keratin materials of at least one layer of a composition as defined above.

Preferably, said cosmetic process is characterized in that it is intended to promote hydration of keratin materials and/or an immediate smoothing effect of keratin materials and/or to promote the firmness and/or elasticity and/or density and/or volume of keratin materials, especially of the skin and/or lips, and thus to prevent and/or reduce the signs of skin aging, in particular to prevent and/or reduce a loss of firmness and/or elasticity, and/or density and/or volume of the skin and/or lips, and/or the appearance of wrinkles, especially on the contour of the lips and/or lines on the lips.

Preferably, said cosmetic process is characterized in that said composition is applied to healthy subjects presenting signs related to aging of the skin and/or lips, in particular a loss of firmness and/or elasticity, and/or density and/or volume of the skin and/or lips, and/or wrinkles of the skin and/or on the contour of the lips and/or thin lips and/or lips presenting lines.

According to another aspect, the present invention also relates to the cosmetic use of at least one cross-linked hyaluronic acid of high molecular weight having a molecular weight greater than or equal to 2000 kDa, preferably greater than or equal to 3000 kDa, or one of its salts, and a low molecular weight non-cross-linked hyaluronic acid having a molecular weight less than or equal to 300 kDa, or one of its salts, as a combination of active ingredients to provide an immediate smoothing effect for keratin materials, especially for the skin and/or lips.

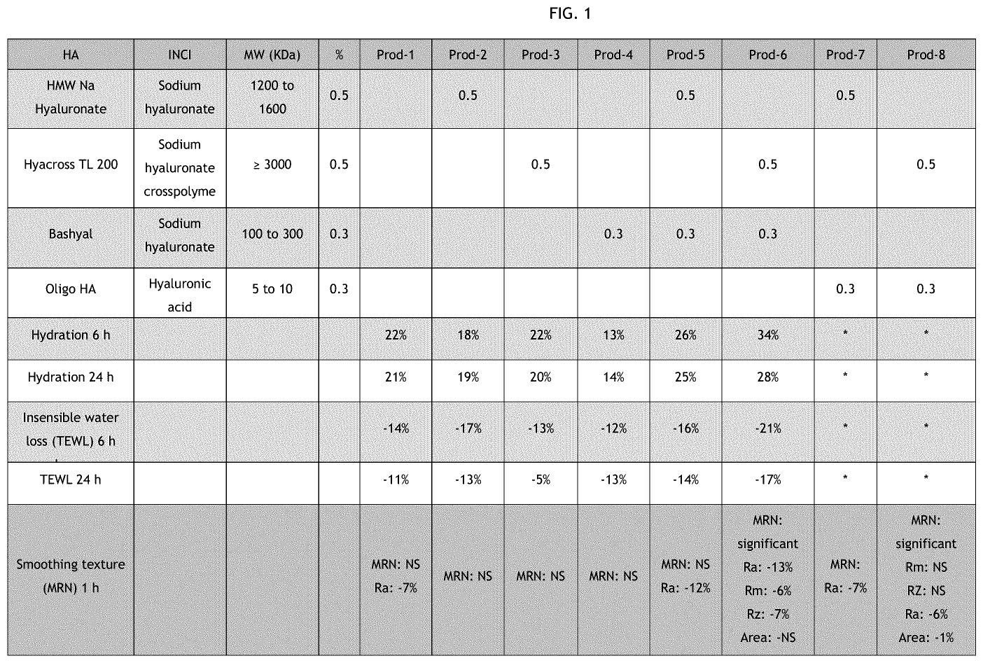

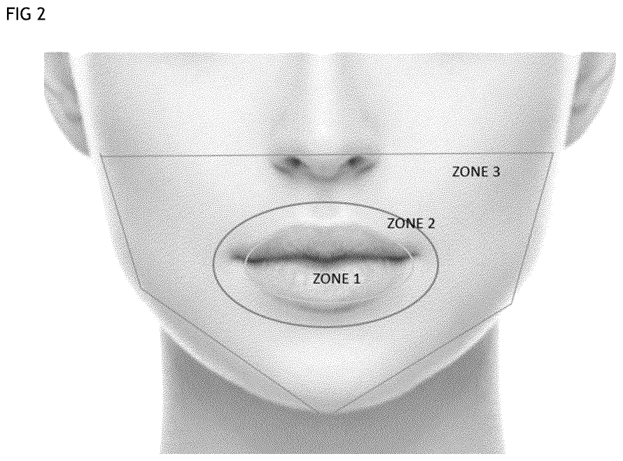

### DESCRIPTION OF THE FIGURES

[FIG. 1] In-vivo effects of the combination of hyaluronic acids according to the invention on hydration and the immediate smoothing effect.

[FIG. 2] Representation of the 3 areas studied during the clinical assessment by the expert panel.

### DETAILED DESCRIPTION OF THE INVENTION

### Combination of Particular Hyaluronic Acids According to the Invention

According to a first aspect, the present invention relates to the combination of at least one high molecular weight cross-linked hyaluronic acid and one low molecular weight non-cross-linked hyaluronic acid.

Surprisingly, the Applicant was able to show that the combination of these two particular hyaluronic acids in the same cosmetic composition produces a synergistic effect, thus reinforcing the benefits provided individually by each of these hyaluronic acids.

According to a particular embodiment, the hyaluronic acids used according to the invention are implemented according to the invention in the form of a powder or a gel in cosmetic compositions.

When hyaluronic acid is in powder form, it is generally present at 100% active ingredient.

When hyaluronic acid is in gel form, it is generally present in a content ranging from 0.1 to 10% by weight of active material relative to the total weight of the gel.

By “hyaluronic acid” or “HA” is meant a polymer formed by the repetition of disaccharide monomers consisting of glucuronic acid and N-acetylglucosamine linked together by alternating B-1,4 and B-1,3 glycosidic bonds. Naturally present in a linear form, the hyaluronic acid macromolecule can also exist in a cross-linked form. By “cross-linked hyaluronic acid” is meant that the chains of hyaluronic acids are linked together by covalent or ionic bonds, thus creating an ordered three-dimensional network. Depending on the different applications, it is possible to produce cross-linked hyaluronic acid with different degrees of cross-linking. The cross-linked polymers based on hyaluronic acids are obtained by methods well known to the person skilled in the art. Typical examples of cross-linkers currently used to produce cross-linked hyaluronic acid are divinyl sulfone (DVS), 1,4-butanediol diglycidyl ether, water-soluble carbodiimides, and cross-linkers containing several amine groups.

By “hyaluronic acid salt” is meant especially sodium hyaluronate, potassium hyaluronate, or any other cosmetically acceptable hyaluronic acid salt.

Hyaluronic acid polymers can be of variable size, especially depending on the number of repetitions and the degree of cross-linking. Typically, hyaluronic acid has a molar mass of between a few thousand Daltons and several million, for example, between 10³ and 10⁷ Da, i.e., between 1 and 10,000 kDa.

### High Molecular Weight Cross-Linked Hyaluronic Acid

The combination of active ingredients according to the invention therefore comprises at least one high molecular weight cross-linked hyaluronic acid or one of its salts.

According to the present invention, the high molecular weight cross-linked hyaluronic acid has a degree of cross-linking ranging from 0.001% to 5%, preferably from 0.01% to 1% and most preferably from 0.1% to 0.6%.

According to the present invention, a hyaluronic acid is referred to as a “high molecular weight hyaluronic acid” when it has a molecular weight greater than or equal to 1000 kDa, preferably greater than or equal to 1200 kDa, preferably greater than or equal to 1400 kDa, preferably greater than or equal to 2000 kDa, such as, for example, greater than or equal to 3000 kDa.

According to the present invention, the molecular weight or molecular mass of hyaluronic acid means the “mean” molecular mass (or mean molecular weight) of the hyaluronic acid molecules constituting the raw material used. Actually, the raw materials used as sources of hyaluronic acids include hyaluronic acid polymers whose molar mass can vary slightly. These variations in molecular mass are especially related to the fact that the number of basic units (i.e., disaccharide monomers) included in the hyaluronic acid molecule at the time of its polymerization varies slightly. So, “molar mass of hyaluronic acid” or “mean molar mass of hyaluronic acid”, must be understood to mean that the molar mass is “approximately” the indicated mass. In particular, the molar mass of hyaluronic acid indicated may vary by ±20%, preferably ±15% and even more preferably ±10%.

Thus, according to the present invention, when we speak of a hyaluronic acid with a molar mass equal to 1000 kDa, is meant that the mean molar mass of said hyaluronic acid is approximately 1000 kDa but that it can be between 800 and 1200 kDa, preferably between 850 and 1150 kDa and preferably between 900 and 1100 kDa.

According to a particular embodiment, the high molecular weight cross-linked hyaluronic acid used in the present invention is in the form of a gel. According to this particular embodiment, the high molecular weight cross-linked hyaluronic acid used in the present invention is preferably the product marketed under the name “Phylcare® Hyacross™ TL 100” by Bloomage Freda Biopharm Co., Ltd. “Phylcare® Hyacross™ TL 100” is a hyaluronic acid gel comprising 1% to 5% by weight of active ingredient relative to the total weight of the product. The hyaluronic acid polymers used in the composition of this product have a molar mass ranging from 1000 kDa to 1800 kDa. The product “Phylcare® Hyacross™ TL 100” is referenced under the International Nomenclature Cosmetic Ingredient (INCI): SODIUM HYALURONATE CROSSPOLYMER, PENTYLENE GLYCOL, AQUA.

According to another particular embodiment, the high molecular weight cross-linked hyaluronic acid used in the present invention is in the form of a powder. According to this particular embodiment, the high molecular weight cross-linked hyaluronic acid used in the present invention is preferably the product with the commercial name “Phylcare® Hyacross™ TL 200” produced by Bloomage Freda Biopharm Co., Ltd. “Phylcare® Hyacross™ TL 200” is a hyaluronic acid in powder form, 100% pure, i.e., composed of 100% active ingredient. The hyaluronic acid polymers used in the composition of this product have a molar mass greater than or equal to 3000 kDa. The product “Phylcare® Hyacross™ TL 200” is referenced under the international nomenclature (INCI): SODIUM HYALURONATE CROSSPOLYMER.

In its capacity as a cosmetic active ingredient, the high molecular weight cross-linked hyaluronic acid is present in the cosmetic composition of the invention in an effective quantity to obtain the desired effect.

In particular, the high molecular weight cross-linked hyaluronic acid, preferably the product with the trade name “Phylcare® Hyacross™ TL 200”, is present in a content ranging from 0.001% to 10%, preferably from 0.005% to 5%, more preferably from 0.01% to 1% by weight of raw material (weight of product) relative to the total weight of the composition.

### Low Molecular Weight Non-Cross-Linked Hyaluronic Acid

The combination of active ingredients according to the invention also comprises at least one low molecular weight non-cross-linked hyaluronic acid or one of its salts.

According to the present invention, hyaluronic acid is referred to as “low molecular weight hyaluronic acid” when it has a molecular weight less than or equal to 500 kDa, preferably less than or equal to 400 kDa, preferably less than or equal to 300 kDa.

According to a particular embodiment, the low molecular weight hyaluronic acid used according to the present invention, in combination, has a molecular weight less than or equal to 500 kDa, preferably less than or equal to 400 kDa, preferably less than or equal to 300 kDa and preferably greater than or equal to 50 kDa, preferably greater than or equal to 100 kDa. In particular, according to a particular embodiment, its molecular weight ranges from 100 kDa to 300 kDa.

According to a particular embodiment, the low molecular weight hyaluronic acid used according to the present invention, in combination, has a molecular weight less than or equal to 200 kDa, preferably less than or equal to 150 kDa, preferably less than or equal to 100 kDa and preferably greater than or equal to 10 kDa, preferably greater than or equal to 50 kDa. In particular, according to a particular embodiment, its molecular weight ranges from 10 kDa to 100 kDa.

According to a particular embodiment, the low molecular weight hyaluronic acid used according to the present invention, in combination, has a molecular weight less than or equal to 100 kDa, preferably less than or equal to 50 kDa, preferably less than or equal to 10 kDa and preferably greater than or equal to 1 kDa, preferably greater than or equal to 5 kDa. In particular, according to a particular embodiment, its molecular weight ranges from 5 kDa to 10 kDa.

According to the present invention, said low molecular weight non-cross-linked hyaluronic acid can be in the form of a gel or in the form of a powder. Advantageously, it is in the form of a powder.

According to a particular embodiment, the low molecular weight non-cross-linked hyaluronic acid used in the present invention is the product marketed under the name “Bashyal powder” by the company Soliance. The “Bashyal powder” product is a hyaluronic acid in powder form, 100% pure, i.e., composed of 100% active ingredient.

The hyaluronic acid polymers used in the composition of this product have a molar mass ranging from 100 kDa to 300 kDa. “Bashyal powder” is referenced under the international nomenclature (INCI): SODIUM HYALURONATE.

According to a particular embodiment, the low molecular weight non-cross-linked hyaluronic acid used in the present invention is the product marketed under the name “PRIMALHYAL ULTRAFILLER” by the company Givaudan. The product “PRIMALHYAL ULTRAFILLER” is a hyaluronic acid in powder form, 100% pure, i.e., composed of 100% active ingredient. The hyaluronic acid polymers used in the composition of this product have a mean molar mass of less than 15 kDa. The “PRIMALHYAL ULTRAFILLER” is referenced under the international nomenclature (INCI): SODIUM ACETYL HYALURONATE.

According to another particular embodiment, the low molecular weight non-cross-linked hyaluronic acid used in the present invention is the product marketed under the name “Oligo HA” by the company Sochibo. The “Oligo HA” product is a hyaluronic acid in powder form, 100% pure, i.e., composed of 100% active ingredient. The hyaluronic acid polymers used in the composition of this product have a molar mass ranging from 5 kDa to 10 kDa. The “Oligo HA” is referenced under the international nomenclature (INCI): HYALURONIC ACID.

In its capacity as a cosmetic active ingredient, the low molecular weight non-cross-linked hyaluronic acid is present in the cosmetic composition of the invention in an effective quantity to obtain the desired effect.

In particular, the non-cross-linked hyaluronic acid of low molecular weight, preferably the product with the trade name “Bashyal powder” or the product with the trade name “Oligo HA”, or the product with the trade name “PRIMALHYAL ULTRAFILLER” is present in a content ranging from 0.001% to 10%, preferably from 0.005% to 5%, more preferably from 0.01% to 1% by weight of raw material (weight of product) relative to the total weight of the composition.

### Cosmetic and Galenic Compositions According to the Invention

In the context of the present invention, the combination of hyaluronic acids described above is used especially for the preparation of cosmetic compositions, in particular cosmetic compositions suitable for topical application on keratin materials, especially on the skin, preferably the skin of the face and/or neck, and/or the lips.

Thus, according to another aspect, the present invention relates to a cosmetic composition comprising, in a physiologically acceptable medium, the combination of at least one high molecular weight cross-linked hyaluronic acid as defined above, or one of its salts, and at least one low molecular weight non-cross-linked hyaluronic acid as defined above, or one of its salts.

By “cosmetic composition” is meant any composition for cosmetic, that is to say, aesthetic purposes, which can be brought into contact with the superficial parts of the human body and more particularly with keratin materials, in particular human skin and/or lips.

By “physiologically acceptable medium” is meant any excipient suitable for topical use, in contact with keratin materials, without risk of toxicity, incompatibility, instability and/or allergic response.

By “keratin materials” according to the invention is meant the skin and/or its appendages, and more particularly human skin and/or lips. In particular, this will concern the skin of the face and/or neck and/or body, and the lips.

According to one embodiment, the composition according to the invention comprises high molecular weight cross-linked hyaluronic acid having the INCI name: SODIUM HYALURONATE CROSSPOLYMER, PENTYLENE GLYCOL, AQUA, in particular the active ingredient marketed under the name “Phylcare® Hyacross™ TL 100” produced by Bloomage Freda Biopharm Co., Ltd.

According to one embodiment, the composition according to the invention comprises high molecular weight cross-linked hyaluronic acid having the INCI name: SODIUM HYALURONATE CROSSPOLYMER, in particular the active ingredient marketed under the name “Phylcare® Hyacross™ TL 200” produced by Bloomage Freda Biopharm Co., Ltd.

According to one embodiment, the composition according to the invention comprises low molecular weight non-cross-linked hyaluronic acid having the INCI name: SODIUM HYALURONATE, in particular the active ingredient marketed under the name “Bashyal powder” by the company Soliance.

According to another embodiment, the composition according to the invention comprises low molecular weight non-cross-linked hyaluronic acid having the INCI name: HYALURONIC ACID, in particular the active ingredient marketed under the name “Oligo HA” by the company Sochibo.

According to a preferred embodiment, the composition according to the invention comprises, in a physiologically acceptable medium:
high molecular weight cross-linked hyaluronic acid, in particular the active ingredient with the trade name “Phylcare® Hyacross™ TL 200” produced by the company Bloomage Freda Biopharm Co., Ltd, with the INCI name: SODIUM HYALURONATE CROSSPOLYMER, and
low molecular weight non-cross-linked hyaluronic acid, in particular the active ingredient with the trade name “Bashyal powder” by the company Soliance, with the INCI name: SODIUM HYALURONATE.

According to another preferred embodiment, the composition according to the invention comprises, in a physiologically acceptable medium:
high molecular weight cross-linked hyaluronic acid, in particular the active ingredient with the trade name “Phylcare® Hyacross™ TL 200” produced by the company Bloomage Freda Biopharm Co., Ltd, with the INCI name: SODIUM HYALURONATE CROSSPOLYMER, and
low molecular weight non-cross-linked hyaluronic acid, in particular the active ingredient with the trade name “Oligo HA” by the company Sochibo, with the INCI name: HYALURONIC ACID.

The cosmetic composition according to the invention comprises said hyaluronic acids, respectively high molecular weight cross-linked hyaluronic acid and low molecular weight non-cross-linked hyaluronic acid, in an effective quantity to obtain the desired effect.

In a particular embodiment, the composition according to the invention comprises said high molecular weight cross-linked hyaluronic acid and said low molecular weight non-cross-linked hyaluronic acid, each in a content ranging from 0.001% to 10%, preferably 0.005% to 5%, more preferably from 0.01% to 1% by weight of raw material (product weight) relative to the total weight of the composition.

According to a particular embodiment, each of the hyaluronic acids used according to the invention is present in the composition in a content ranging from 0.05% to 0.8% by weight, more preferably from 0.1 to 0.6% by weight of raw material (product weight) relative to the total weight of the composition.

When the hyaluronic acids are dispersed in a gel-forming solvent, they are each present in the composition in a content ranging from 0.1% to 10% by weight, more preferably from 1% to 5% by weight of active material (hyaluronic acid polymer) relative to the total weight of the composition.

Advantageously, said cosmetic composition according to the invention comprises said high molecular weight cross-linked hyaluronic acid and said low molecular weight non-cross-linked hyaluronic acid in a high molecular weight cross-linked hyaluronic acid/low molecular weight non-cross-linked hyaluronic acid ratio (by weight of raw material) comprised between 0.01 and 100, preferably between 0.1 and 10, most preferably between 0.5 and 2.

Reasoning by weight of active material of hyaluronic acid polymer, the ratio of the high molecular weight cross-linked hyaluronic acid to said low molecular weight non-cross-linked hyaluronic acid will generally range from 0.1 to 10, in particular from 0.5 to 2.

In one embodiment, the cosmetic composition of the invention is a care and/or makeup composition for keratin materials, in particular for the skin, preferably the skin of the face and/or neck, and/or the lips.

According to a preferred embodiment, the cosmetic composition of the invention is a composition for caring for keratin materials, in particular for the skin, preferably the skin of the face and/or neck, and/or the lips.

The cosmetic composition of the invention comprises at the least high molecular weight cross-linked hyaluronic acid and low molecular weight non-cross-linked hyaluronic acid, as defined above, and, in addition, one or more acceptable cosmetic excipients among those known to the person skilled in the art with a view to obtaining a composition for topical application.

The invention therefore also relates to a composition, in particular a cosmetic composition in the form of a topical composition for the skin and/or lips, in particular in the form of a cream, oil-in-water or water-in-oil emulsion or multiple emulsion, suspension, gel, milk, lotion, serum, mask or lip balm.

The composition of the invention may also further comprise at least one cosmetic adjuvant chosen from the group consisting of antioxidant agents, emollient agents, moisturizing agents, anti-aging agents, fragrances, and mixtures thereof.

The cosmetic adjuvants advantageously used in the composition of the invention may be present in a content ranging from 0.0001% to 10%, preferably from 0.001% to 5% by weight relative to the total weight of the composition.

According to certain embodiments, the composition of the invention may advantageously comprise one or more cosmetic ingredients comprising one or more polyglycerols having from 3 to 10 glycerol units, preferably chosen from the products of INCI name polyglycerin-3 and polyglycerin-6.

As cosmetic ingredients based on polyglycerols usable according to the invention, the references marketed by the company SAKAMOTO can especially be named
PGL-S of INCI name POLYGLYCERIN-3
POLYGLYCERIN 310 of INCI name POLYGLYCERIN-4
POLYGLYCERIN-500 MB of INCI name POLYGLYCERIN-6 and AQUA (WATER)
POLYGLYCERIN-750 MB of INCI name POLYGLYCERIN-10 and AQUA (WATER).

The Applicant has actually been able to show that the use of polyglycerols, especially in combination with at least one high molecular weight cross-linked hyaluronic acid as defined above, or one of its salts, and at least one low molecular weight non-cross-linked hyaluronic acid as defined above, or one of its salts, makes it possible to further improve the plumping effect of the lips, and especially the immediate and/or long-term plumping effect

According to one embodiment, the cosmetic composition of the invention comprises at least 0.5% polyglycerols, in particular at least 1% polyglycerols, preferably at least 2% polyglycerols, in particular chosen from the products of INCI name polyglycerin-3 and polyglycerin-6.

According to one embodiment, the cosmetic composition of the invention comprises less than 10% of polyglycerols, in particular less than 8% of polyglycerols, preferably less than 6% of polyglycerols, in particular chosen from products with the INCI name polyglycerin-3 and polyglycerin-6.

Depending on the nature of the composition, one or more cosmetically acceptable excipients can also be selected. All excipients commonly used in the preparation of cosmetic compositions intended for use on keratin materials can be used in the present invention. Preferably, the cosmetically acceptable excipients are selected from polymers, surfactants, rheology agents, electrolytes, pH adjusters, preservatives, dyes, nacres, pigments and mixtures thereof.

According to a particular embodiment, the cosmetic composition of the invention comprises less than 5% of filler, in particular less than 2% of filler, preferably less than 1% of filler, in particular of filler with a blurring effect.

However, according to another particular embodiment, the cosmetic composition of the invention is devoid of filler, in particular filler with a blurring effect. Indeed, while fillers with a blurring effect advantageously make it possible to obtain an immediate smoothing effect, gain viscosity and have a less shiny appearance upon application, these fillers can negatively impact the hydrating effect of hyaluronic acid.

“Fillers” mean particles of plate, spherical or oblong shape, colourless or white, mineral or organic, natural or synthetic, which are in an insoluble form and dispersed in the medium of the composition. These fillers especially serve to modify the rheology or texture of the composition and/or to confer a mattifying effect. “Blurring effect” filler means an effect which camouflages the micro-reliefs of the skin and thus makes it possible to attenuate, through an optical correction effect, imperfections in relief and/or colour, such as wrinkles, fine lines, pores and spots.

### Cosmetic Processes and Uses

The cosmetic processes and cosmetic uses described below are intended for application to keratin materials, especially healthy keratin materials, preferably skin, in particular the skin of the face and/or neck, and healthy lips of “healthy” subjects.

The keratin materials according to the invention are especially healthy keratin materials (“healthy” subjects), i.e., not presenting conditions or disorders which would relate to a pathological state (“unhealthy” subjects, affected with a pathology). The terms healthy skin and/or lips or skin and/or lips will be used interchangeably in the rest of the description.

Another object of the invention relates to a cosmetic process for caring for keratin materials, especially healthy keratin materials, in particular the skin and/or lips, comprising the application to said keratin materials of at least one layer of a cosmetic composition as described above.

In the context of the present invention, said cosmetic process is intended to promote hydration of keratin materials, especially of the skin and/or lips.

In the context of the present invention, said cosmetic process is also intended to provide an immediate smoothing effect on keratin materials, especially on the skin and/or lips.

By “smoothing effect” is meant that the composition according to the invention makes it possible to reduce skin roughness and/or microrelief. By “immediate smoothing effect” is meant that the composition according to the invention makes it possible to obtain a smoothing effect within one hour after application of said composition.

Thus, especially through its effects on hydration, roughness, and skin microrelief, said cosmetic process of the invention makes it possible to reduce the signs of skin aging.

In the context of the present invention, said cosmetic process is also intended to promote the firmness and/or elasticity of keratin materials, especially of the skin and/or lips, and thus prevent and/or reduce the signs of skin aging, in particular to prevent and/or reduce the appearance of wrinkles.

Thus, the cosmetic process of the invention also has particularly interesting anti-aging properties.

According to another aspect, the present invention relates to the cosmetic use of at least one high molecular weight cross-linked hyaluronic acid and one low molecular weight non-cross-linked hyaluronic acid as defined previously as a combination of active ingredients to provide an immediate smoothing effect on keratin materials, especially on the skin and/or lips.

According to a particular and preferred embodiment, the compositions used in the cosmetic processes and uses according to the invention comprise:
high molecular weight cross-linked hyaluronic acid, in particular the active ingredient with the trade name “Phylcare® Hyacross™ TL 200” produced by the company Bloomage Freda Biopharm Co., Ltd, with the INCI name: SODIUM HYALURONATE CROSSPOLYMER,
and
low molecular weight non-crosslinked hyaluronic acid, in particular the active ingredient with the trade name “Bashyal powder” by the company Soliance, with the INCI name: SODIUM HYALURONATE.

According to another particular and preferred embodiment, the compositions used in the cosmetic processes and uses according to the invention comprise:
high molecular weight cross-linked hyaluronic acid, in particular the active ingredient with the trade name “Phylcare® Hyacross™ TL 200” produced by the company Bloomage Freda Biopharm Co., Ltd, with the INCI name: SODIUM HYALURONATE CROSSPOLYMER,
and
low molecular weight non-cross-linked hyaluronic acid, in particular the active ingredient with the trade name “Oligo HA” by the company Sochibo, with the INCI name: HYALURONIC ACID.

According to a particular embodiment, the compositions of the invention will be applied to healthy subjects with dry skin.

According to a particular embodiment, the compositions of the invention will be applied to healthy subjects presenting a loss of firmness and/or elasticity of the skin.

According to another particular embodiment, the compositions of the invention will be applied to healthy subjects presenting signs related to skin aging, in particular wrinkles.

According to a particular embodiment, the compositions of the invention will be applied to healthy subjects having dry lips and/or thin lips and/or lips presenting lines and/or wrinkles on the contour of the lips.

The present invention will now be illustrated in the following non-limiting examples. The % are expressed by weight of raw material relative to the total weight of the composition, unless otherwise indicated.

### EXAMPLES

### Example 1: Study of the Immediate Smoothing Effect of the Skin

### 1-1 Study Protocol

### Study Purpose

The purpose of the study is to evaluate and compare the immediate smoothing effect on the microrelief network or MRN (before and 1 hour after application) of one or more products by the skin replica method.

The immediate smoothing effect of the different products was evaluated on male or female volunteers, all above age 35, and without skin disease in the area evaluated.

### Measurement Principle

Replicas of the skin surface are taken at different times during the study. These replicas are imprints of the surface of the skin which are then used to evaluate the cosmetic effects of the active ingredients tested. These replicas are processed by image analysis using Quantilines software.

The replicas are taken by applying 1 adhesive ring of 24 mm in diameter to the test area. Then using a spatula, Silflo silicone casting resin (Flexico) plus catalyst (Flexico) is spread inside the ring. After drying, the replica is then peeled off from the surface of the skin.

The replica is illuminated by a grazing illuminator at an angle of incidence of 35 to generate shadows behind each line of the skin's surface. These shadows are then analysed on the video images of the replica using Quantilines software, which calculates parameters representative of the skin surface.

The replicas are positioned in relation to the light source so as to maximize shading (north-south orientation).

The replicas taken after treatment are strictly repositioned relative to those taken before treatment.

The surface of the replica is divided by 90 equidistant lines, for each of which the software calculates the following parameters:
Ra is the mean roughness, the ratio of the integrated area of the length profile to the length of the profile. Mean Ra is the arithmetic mean of the Ra values of all profiles (in arbitrary units).
Rz is the mean amplitude of the peak on the profile considered. Each profile is divided into 5 segments of equal length for each of which the amplitude of the peak Rzi is calculated. Mean Rz is the arithmetic mean of the Rzi values of all profiles (in arbitrary units).
Rm is the maximum roughness (the maximum roughness between the 5 segments analysed).
Area: corresponds to the factor between the actual length of the skin and that which can be seen. This is the smoothing of the skin and highlighting of micro-wrinkles. The more micro-wrinkles the skin has, the more the area increases. The result is displayed in the “Area” result window in %.

The measurements were taken on the skin of the forearm, before application of the product evaluated and then 1 hour after application. The product evaluated was spread by a technician in an amount of 2 μL/cm².

### Endpoints

The main endpoints are Rm, Rz, Ra and Area.

These parameters decrease if the treatment has a beneficial effect on skin smoothing. The product can be said to have a smoothing effect on the skin if at least two of the primary endpoints significantly decrease.

The difference between the value before and the value one hour after application is calculated for each parameter, each volunteer and each product. These differences are compared by analysis of variance. If they are significant, the products are classified using the Newman-Keuls test.

### Statistical Analyses

The statistical method used is analysis of variance (ANOVA). It was assumed that the data follow the normal distribution. With ANOVA, the Student-Newman-Keuls test is performed for multiple comparisons of means and determination of resemblance group.

Sample size: A sufficient number of volunteers must be included to allow the evaluation of 12 subjects.

Level of significance: α risk less than or equal (< or =) to 5%.

### Calculation Methods:

Effect of the product on the skin:
For each parameter and each product, calculation and comparison by ANOVA of the means, before treatment (t₀) and the times after treatment (tᵢ).
The percentage change is calculated using the following equation:

$${VAR}_{i} \% = ( {P}_{i} - {P}_{0} ) / {{P}_{0}}^{*} 100$$

wherein:
P₀: mean of the parameter of the treated area at time t₀.
Pᵢ: mean of the parameter of the treated area at time tᵢ.
Percentages are calculated only if the differences are significant compared to the pre-treatment values.

### 1-2: Commercial References of the Cosmetic Active Ingredients Used in the Examples

### High Molecular Weight Hyaluronic Acid

“Hyacross TL200”
“Phylcare® Hyacross™ TL 200” commercial product produced by the company Bloomage Freda Biopharm Co., Ltd, whose INCI name is: SODIUM HYALURONATE CROSSPOLYMER.
Cross-linked hyaluronic acid of molecular weight greater than or equal to 3000 kDa.
“HMW Na hyaluronate”
“Bio-sodium hyaluronate powder” commercial product produced by the company Sochibios, with the INCI name: SODIUM HYALURONATE.
Non-cross-linked hyaluronic acid of mean molecular weight ranging from 1000 kDa and 1500 kDa.

### Low Molecular Weight Non-Cross-Linked Hyaluronic Acid

“Bashyal”
“Bashyal powder” commercial product produced by the company Soliance, with the INCI name: SODIUM HYALURONATE.
Hyaluronic acid of mean molecular weight ranging from 100 kDa to 300 kDa.
“Oligo HA”
“Oligo HA” commercial product produced by the company Sochibo, with the INCI name: HYALURONIC ACID.
Hyaluronic acid of mean molecular weight ranging from 5 kDa to 10 kDa.

Whether alone or in combination, these active ingredients were used in the examples at an individual concentration ranging from 0.3% to 0.5% by weight of raw material relative to the total weight of the composition.

### 1-3: Formulations Evaluated

TABLE 1

| | Content |
|---|---|
| Placebo Composition | (%) |
| Water | QS 100 |
| Glycols | 12 |
| Carbomer (Carbopol Ultrez 20) | 0.03 to 0.35 |
| Preservatives | 0.27 |

Different hyaluronic acids, cross-linked or non-cross-linked, of high and low molecular weight were tested, alone and in combination, in the formula described above.

TABLE 2

| Formula | | |
|---|---|---|
| No. | Test | Composition of the formula evaluated |
| Prod-1 | Comparison test | Placebo |
| | | (without hyaluronic acid) |
| Prod-2 | Comparison test | Placebo + |
| | | 0.5% non-cross-linked HMW Na |
| | | Hyaluronate (high MW non-cross-linked) |
| Prod-3 | Comparison test | Placebo + |
| | | 0.5% cross-linked Hyacross |
| | | TL200 (high MW cross-linked) |
| Prod-4 | Comparison test | Placebo + |
| | | 0.3% Bashyal (low MW non-cross-linked) |
| Prod-5 | Comparison test | Placebo + |
| | | 0.5% non-cross-linked HMW Na |
| | | Hyaluronate + 0.3% Bashyal |
| Prod-6 | According to the | Placebo + |
| | invention | 0.5% cross-linked Hyacross TL |
| | | 200 + 0.3% Bashyal |
| Prod-7 | Comparison test | Placebo + |
| | | 0.5% non-cross-linked HMW Na |
| | | Hyaluronate + 0.3% Oligo HA |
| Prod-8 | According to the | Placebo + |
| | invention | 0.5% cross-linked Hyacross |
| | | TL200 + 0.3% Oligo HA |

The placebo is a simplex formulation composed of water, glycols and preservatives. To obtain similar viscosities among the different tests, a viscosity agent (Carbopol Ultrez 20=acrylates/C10-30 alkyl acrylate crosspolymer neutralized to pH around 6 with sodium hydroxide) was added. The amount of viscosifier was adjusted in the different formulas to maintain an equivalent viscosity.

### Formula Preparation Process

a. Heat phase A to 80° C. (water+preservatives);
b. Add Carbopol Ultrez 20 into phase A with deflocculation at 300 RPM until the particles disappear;
c. Add a 10% sodium hydroxide solution into phase A at 500 RPM until a gel forms;
d. Weigh the glycols (Glycerine/Butylene glycol/propane diol/pentylene glycol) into a capsule;
e. Wet the hyaluronic acid(s) (HA) in the glycol mixture until a homogeneous paste is formed;
f. Add the glycol/HA mixture obtained in step (e) into phase A obtained in step (c) at 60° C. with deflocculation at 600 RPM;
g. Cool with deflocculation at 400 RPM to 30° C.;
h. Stop the motor and water compensation.

### 1-4: Results of the Study on the Immediate Smoothing Effect

The results of this study are presented in FIG. 1.

Surprisingly, it has been observed that the particular combination of certain types of hyaluronic acids produces a synergistic effect on the immediate smoothing effect.

In particular, the combination of a low molecular weight hyaluronic acid such as “Bashyal” or “Oligo HA”, with a high molecular weight cross-linked hyaluronic acid such as “Hyacross TL 200” makes it possible to obtain significant immediate smoothing results.

Moreover, it has been observed that the combination of these same low molecular weight hyaluronic acids with a high molecular weight non-cross-linked hyaluronic acid such as “HMW Na Hyaluronate” does not make it possible to obtain this immediate smoothing effect.

These results therefore show that, surprisingly, the cross-linking of the high molecular weight hyaluronic acid polymer is essential to obtain an immediate smoothing effect.

### Example 2: Study of Skin Hydration

### 2-1 Study Protocol

### Study Purpose

The purpose of the study is to evaluate and compare the short-term moisturizing effect (6 hours and 24 hours) of one or more products by measuring the variation in electrical capacitance and impedance of the skin using a corneometer.

The moisturizing effect of the different products was evaluated on male or female volunteers, all aged at least 18 years, and without skin disease in the area evaluated.

### Measurement Principle

The corneometer measures the variation in electrical capacitance as a function of the water content of the skin (variation in the dielectric constant of the skin). Its sensor consists of two electrodes, which are used to generate an electric field on the skin surface. The capacitance of the electrode system and the skin is influenced by changes in the dielectric constant of the biological medium in contact with the probe. The more hydrated the skin, the higher the dielectric constant. An alternating current is applied to the electrodes. The time required to reach a given potential difference is inversely proportional to the capacitance of the system, and an increase in the dielectric constant of the stratum corneum is recorded by the corneometer as an increase in the frequency of the alternating current. Capacitance changes are expressed in arbitrary units.

The impedance indicates the degree of hydration of the upper layers of the epidermis. The dielectric constant of the skin is related to the water content of the skin. The measurement of electrical properties is recognized as an objective method for assessing the hydration of the stratum corneum (E. Berardesca-EEMCO guidance for the assessment of stratum corneum hydration: electrical methods-Skin Research and Technology 1997; 3:126-132).

The corneometer determines the skin capacitance, Cx, which is part of the formula defining impedance:

$$Z = {[ {Rx}^{2} + {( 1 / 2 π fCx )}^{2} ]}^{1 / 2}$$

Wherein: Z: impedance
   Rx: resistance (resistance=1/conductance)
   f: alternating current frequency
   Cx: capacitance

The measurements are done on the forearms. The areas are protected from friction by rings held by a hypoallergenic, stretchable, non-occlusive adhesive fabric (Hypafix from Smith & Nephew) between each measurement and are removed just before the 15-minute stabilization period.

Three successive measurements are done with the corneometer after 15 minutes of stabilization, which makes it possible to adjust the area to the ambient conditions of the measuring room (temperature: 22° C.+/−2° C. and relative humidity: 50%+/−10%).

There is no prior preparation of the skin. The tested product is applied by a technician before the start of measurements, all at once, in an amount of 2 μL per cm².

On the morning of the study (before the measurements), the volunteers used only their usual personal care products. No other products should be used during the study.

### Endpoint

Electrical capacitance is expressed in arbitrary units on a scale of approximately 0 to 150. The higher the value, the greater the hydration.

### Statistical Analyses

The statistical method used is analysis of variance (ANOVA). It was assumed that the data follow the normal distribution. With ANOVA, the Student-Newman-Keuls test is performed for multiple comparisons of means and determination of resemblance group.

Sample size: A sufficient number of volunteers must be included to allow the evaluation of 10 subjects.

Level of significance: α risk less than or equal (< or =) to 5%.

### Calculation Methods:

The untreated area is considered a product.
The factors of the analysis are: the volunteers, the time of measurement and the products.
1—Overall ANOVA, all factors combined on raw data. The residual graph helps detect outliers.
2—Calculate the mean values of the 3 measurements: grouping of the means.
3—Overall ANOVA, all factors combined on the means for validation of the grouping (comparison with the means of 1−).
4—ANOVA on the mean values for all products & volunteers combined at T₀ (before application) to compare the measurement sites at T₀.
5—Calculate the changes compared to the value before application: T₁−T₀ (=ΔT) With:
T₁: hydration at tᵢ
T₀: hydration of the same area at t₀
6—ANOVA on ΔT, all products & volunteers combined for each measurement time t₀ compare the product at each time.
7—Overall ANOVA on ΔT, all factors together (without T₀) to globally compare the products all together. A product may not be considered in the analysis to more precisely compare other products.
8—

### a) Calculation of Percentages to Compare the Effect of Different Products in Relation to the Untreated Area:

Only if the differences compared to the untreated area (or the reference) are significant.

The percentage is calculated for each volunteer:

$${VAR}_{i} \% = \frac{( {Tr}_{i} - {Tr}_{0} ) - ( {Tnt}_{i} - {Tnt}_{0} )}{{Tnt}_{i}} \times 100$$

With:
   Trᵢ: hydration of the treated area at tᵢ
   Tr₀: hydration of the treated area at t₀
   Tntᵢ: hydration of the untreated control at tᵢ
   Tnt₀: hydration of the untreated control at t₀
      b) Calculation of the Percentages of the Results without the Untreated Area:

The percentage change is calculated as follows from the means:

$${VAR}_{i} \% = ( {T}_{i} - {T}_{0} ) / {{T}_{0}}^{*} 100$$

With:
T₀: hydration at the time “before application”.
Tᵢ: hydration at time tᵢ

Percentages are only calculated if the differences are significant compared to the time “before application”.
9-Overall ANOVA on the percentages to obtain the means in %.

### 2-2: Commercial References of the Cosmetic Active Ingredients Used in the Examples

The commercial references used in this example are the same as in Example 1-2 above.

### 2-3: Formulations Evaluated

The formulations evaluated in this example are the same as in Example 1-3 above.

### 2-4: Results of the Study on the Moisturizing Effect

The results of this study are presented in FIG. 1.

Surprisingly, it has been observed that the particular combination of certain types of hyaluronic acids produces a synergistic effect on hydration.

### Hydration at 6 and 24 Hours

The results show that for the placebo composition and the compositions comprising only one hyaluronic acid “Bashyal” or “Hyacross TL 200” (comparative), the hydration measured is comprised between 13% and 22% at 6 hours and comprised between 14% and 21% at 24 hours (FIG. 1).

Surprisingly, the results show that for the composition comprising the “Bashyal”+ “Hyacross TL 200” combination (according to the invention), the hydration is 34% at 6 hours (vs. 13 to 22% for the comparisons) and 28% at 24 hours (vs. 14 to 21% for comparisons) (FIG. 1).

The combination of a low molecular weight hyaluronic acid such as, for example, “Bashyal” with a high molecular weight cross-linked hyaluronic acid such as “Hyacross TL 200” (according to the invention) makes it possible to obtain an increase in hydration.

Moreover, it has been observed that the combination of “Bashyal”, with a non-cross-linked hyaluronic acid of high molecular weight such as “HMW Na Hyaluronate” (comparative) does not allow such an increase to be obtained.

### Insensible Water Loss (TEWL) at 6 and 24 Hours

The results show that for the placebo composition and the compositions comprising only one hyaluronic acid “Bashyal” or “Hyacross TL 200” (comparative), the insensible water loss measured is comprised between −12% and −14% at 6 hours and comprised between −5% and −13% at 24 hours (FIG. 1).

Surprisingly, the results show that for the composition comprising the “Bashyal”+ “Hyacross TL 200” combination (according to the invention), the imperceptible water loss is −21% at 6 hours and −17% at 24 hours (FIG. 1).

The combination of a low molecular weight hyaluronic acid such as “Bashyal” with a high molecular weight cross-linked hyaluronic acid such as “Hyacross TL 200” (according to the invention) makes it possible to reduce insensible water loss.

Moreover, it has been observed that the combination of “Bashyal”, with a non-cross-linked hyaluronic acid of high molecular weight such as “HMW Na Hyaluronate” (comparative) does not allow such an effect to be obtained.

### CONCLUSION

These results therefore show that, surprisingly, in the context of a combination of a low molecular weight hyaluronic acid and a high molecular weight hyaluronic acid, the cross-linking of the high molecular weight hyaluronic acid polymer is essential to obtain an improved moisturizing effect.

### Example 3: Studies on the Plumping and Smoothing Effect of the Lips

### 3-1 Study Protocol

### Study Purpose

The purpose of this study was to evaluate to what extent the combination of hyaluronic acids according to the invention can provide a plumping and smoothing effect on the lips, the lip contour and the nasolabial area. To this end, the effects of a test formula comprising the combination of a high molecular weight cross-linked hyaluronic acid and a low molecular weight non-cross-linked hyaluronic acid was evaluated on a panel of subjects. The formulas are described below.

### Subjects Included in the Study

The effects were evaluated on female volunteers of European ancestry. These volunteers, or subjects, were divided into two groups depending on the formula to be tested, test formula or control formula.
Group 1:17 volunteers, mean age: 45.6+/−6.7 years old (between 37 and 57 years old).
Group 2:15 volunteers, mean age: 47.5+/−4.9 years old (between 39 and 56 years old).

The volunteers had all skin types (no selection on skin type) and were all users of lip care products. In addition, the volunteers included in this study all exhibited:
wrinkles around the lip contour,
a lack of volume in the lips, and
lips that appear poorly hydrated.

### Formulas Evaluated to Test the Effect of the Combination of Hyaluronic Acids According to the Invention

The formulas tested correspond to a basic cosmetic formulation making it possible to evaluate the cosmetic effectiveness of the active ingredients of interest. This basic formula, also called control composition, is described in Table 3.

Table 3: Basic formula, also called control composition

TABLE 3

| | Content |
|---|---|
| Control composition | (%) |
| Water | QS 100 |
| Vegetable oil | 38 |
| Hydroxystearic acid | 1.2 |
| Gelling agent (bentone) | 13 |
| Vegetable wax | 5 |
| Cellulose | 2 |
| Glycols | 10 |
| Carbopol | 0.2 |
| Preservatives | 0.3 |

During this study, group 2 received the control formula (CTL formula) corresponding to the basic cosmetic formula alone (Table 3), therefore not containing the combination of hyaluronic acids according to the invention.

In return, group 1 received the test composition (2HA formula), i.e., a basic cosmetic formula also containing the combination of hyaluronic acids according to the invention:
“Hyacross TL200”
“Phylcare® Hyacross™ TL 200” commercial product produced by the company Bloomage Freda Biopharm Co., Ltd, whose INCI name is: SODIUM HYALURONATE CROSSPOLYMER. Cross-linked hyaluronic acid of mean molecular weight greater than or equal to 3000 kDa.
“PRIMALHYAL ULTRAFILLER”
Commercial product “PRIMALHYAL ULTRAFILLER” produced by the Givaudan company. Hyaluronic acid with a mean molecular weight of less than 15 kDa.

Table 4: 2HA formula, also called test composition

TABLE 4

| Test composition (according | Content |
|---|---|
| to the invention) | (%) |
| Water | QS 100 |
| Vegetable oil | 38 |
| Hydroxystearic acid | 1.2 |
| Gelling agent (bentone) | 13 |
| Vegetable wax | 5 |
| Cellulose | 2 |
| Glycols | 10 |
| Carbopol | 0.2 |
| Hyacross TL200 | 0.5 |
| PRIMALHYAL ULTRAFILLER | 0.3 |
| Preservatives | 0.3 |

The formulas evaluated (control formula or 2HA formula) were applied to the lips and lip contour twice a day, in the morning and in the evening, for the duration of the study, i.e. 28 days.

### 3-2: Results

### 3.2.1. Study 1

The plumping effect of the formulations tested (described in section 2-1 above) was evaluated by a panel of experts immediately after application, then at 8 hours and 24 hours after application, and finally on day 7 and on day 28 of the study.

The plumping effect was assessed by measuring the curvature of the upper lip.

The results show that the 2HA formula significantly reduces the curvature of the upper lip immediately after application. The decrease in this curvature reflects the increase in lip volume. In addition, and unlike the control formula, this effect is maintained over time since we still see a significant effect at 8 hours and 24 hours versus a non-significant effect for the control formula). This effect is also observed on day 7 and 28 for the 2HA formula only.

Thus, the combination of hyaluronic acids according to the invention advantageously makes it possible to act on plumping the lips upon application with a proven long-term effect.

### 3.2.2. Study 2—Clinical Assessment

As part of this study, a clinical assessment was performed by a trained assessor. The effects of the cosmetic formulations tested (described in paragraph 2-1 above) were evaluated on 3 areas of the face (all including the lips). The criteria evaluated are: the appearance of the lip area (zone 1), the appearance of the lip contour area (zone 2), and the appearance of the broad area surrounding the lips (zone 3). Zones 1, 2 and 3 are shown in FIG. 2.

TABLE 5

| | T4W/T0 | |
|---|---|---|
| | 2HA formula | CTL formula |
| Zone 1: Lip zone | ++ | + |
| Smooth appearance of the lip relief | | |
| Soft appearance of the lips | +++ | NS |
| Plump appearance of the lips | ++ | + |
| Lip health (pink colour) | +++ | ++ |
| Zone 2: Lip contour | +++ | +++ |
| Smooth appearance of wrinkles around the | | |
| lips | | |
| Zone 3: Large area around the lips | +++ | ++ |
| Lifted appearance of the skin | | |
| Distance between nose and lips (lip lift and | ++ | + |
| cupid's bow) | | |
| Smooth appearance of the skin | +++ | ++ |

T4W = 4 weeks after the start of the study;
T0 = at the start of the study, before the first application of the products tested,
NS = Not Significant

Compared to the control formula, it is noted that the 2HA formula makes it possible to obtain better results for all the criteria assessed.

These results reflect the cosmetic interest of the combination of hyaluronic acids according to the invention. This combination advantageously makes it possible to obtain a smoothing and plumping effect on the lips and the area surrounding them, there is especially an improvement in the smooth, soft and plump appearance of the lips and an improvement in the health of the lips (pink colour). A smoothing of wrinkles around the lips and more generally of the skin around the lips is also noted.

### 3-3: Conclusions

These studies, aimed at evaluating the plumping and smoothing effect of cosmetic compositions comprising the combination of hyaluronic acids according to the invention, on the lips and the area of the face surrounding the lips show that the combination of hyaluronic acids according to the invention allows the 2HA formula to be more effective than the control formula with long-term action on smoothing, softness and plumping.

### Example 4: Study by Immunostaining of the Effect of the Combination of Hyaluronic Acids According to the Invention on the Expression of Fibrillin-1

### 4-1: Explant Preparation

24 explants of 12±1 mm in diameter were prepared from an abdominoplasty of a 62-year-old woman of European ancestry with phototype II-III. The explants were placed in BEM (BIO-EC's Explants Medium) at 37° C. in a humid atmosphere, enriched with 5% CO2.

The explants were divided into 6 batches as follows:

TABLE 6

| Batch | Designation/Treatment | Number of explants | Stop |
|---|---|---|---|
| T0 | Plastic surgery controls | 3 | Day 0 |
| T | Untreated controls | 3 | Day 7 |
| P1 | Product P1 | 3 | Day 7 |
| P2 | Product P2 | 3 | Day 7 |
| P3 | Product P3 | 3 | Day 7 |
| P4 | Product P4 | 3 | Day 7 |

### Application of Products

On day 0, day 2, day 5 and day 6, the products tested P1 to P4 were applied topically, in an amount of 2 μL per 1 cm² explant (˜ 2 mg/cm²) and spread using a small spatula. The explants from the control batches did not receive any treatment, except renewal of the medium. Half of the medium was renewed (1 mL/well) on day 1, day [?] and day 5.

### Samples

On day 0, the 3 explants from batch TO were taken and cut in half. Each sample was cut in half, one half was frozen at −80° C. in OCT, the other half was fixed in formalin. On day 7, 3 explants from each batch were taken and treated in the same way as on day 0.

### 4-2: Commercial References of the Cosmetic Active Ingredients Used in the Examples

The commercial references used in this example are the same as in Example 1-2 above.

### 4-3: Formulations Evaluated

TABLE 7

| | Content |
|---|---|
| Placebo Composition | (%) |
| Water | QS 100 |
| Glycols | 12 |
| Carbomer (Carbopol Ultrez 20) | 0.03 to 0.35 |
| Preservatives | 0.27 |

Different hyaluronic acids, cross-linked or non-cross-linked, of high and low molecular weight were tested, alone and in combination, in the formula described above.

TABLE 8

| Formula | | Composition of the |
|---|---|---|
| No. | Test | formula evaluated |
| P1 | Comparison test | Placebo |
| | | (without hyaluronic acid) |
| P2 | Comparison test | Placebo + |
| | | 0.5% cross-linked Hyacross |
| | | TL200 (high MW cross-linked) |
| P3 | Comparison test | Placebo + 0.3% Bashyal |
| | | (low MW non-crosslinked) |
| P4 | according to the | Placebo + |
| | invention | 0.5% cross-linked Hyacross |
| | | TL200 (high MW cross-linked) + |
| | | 0.3% Bashyal (low MW |
| | | non-cross-linked) |

The placebo is a simplex formulation composed of water, glycols and preservatives. To obtain similar viscosities among the different tests, a viscosity agent (Carbopol Ultrez 20=acrylates/C10-30 alkyl acrylate crosspolymer neutralized to pH around 6 with sodium hydroxide) was added. The amount of viscosifier was adjusted in the different formulas to maintain an equivalent viscosity.

### 4-4: Results of the Study on the Expression of Fibrillin-1

### Fibrillin-1 Immunostaining

Fibrillin-1 was labelled on frozen sections with a polyclonal anti-fibrillin-1 antibody (Sima Aldrich,) diluted 1/200 in 0.3% PBS-BSA overnight at room temperature and revealed in AlexaFluor AF488 (Lifetechnologies). The nuclei were counterstained with propidium iodide. Immunostaining was evaluated by microscopic observation and semi-quantified by image analysis.

The results obtained show that treatments with the placebo formula (P1) or with formulas containing only one of the two hyaluronic acids used according to the invention (P2 and P3) do not lead to any significant change in the expression of the fibrillin-1 on day 7.

In contrast, and surprisingly, it has been observed that the combination of a low molecular weight hyaluronic acid such as “Bashyal” with a high molecular weight cross-linked hyaluronic acid such as “Hyacross TL 200”, according to the invention (formula P4) significantly increases (±35%) the expression of fibrillin-1 on day 7. The combination of active ingredients according to the invention therefore makes it possible to produce a synergistic effect on the expression of fibrillin-1.

These results therefore show that the combination of a low molecular weight hyaluronic acid with a high molecular weight cross-linked hyaluronic acid according to the invention can be used to promote firmness and/or elasticity of the skin.

### Example 5: Cosmetic Formulations According to the Invention

The compositions are prepared according to conventional methods used in the cosmetic field.

### Facial Plumper

TABLE 9

| INGREDIENTS | % |
|---|---|
| PURIFIED WATER | QS 100 |
| CHLORPHENESIN | 0.2700 |
| CARBOPOL ULTREZ 20 | 0.0700 |
| 10% SODIUM HYDROXIDE SOLUTION | 0.2100 |
| LW GLYCEROL VEGETAL | 3.0000 |
| 1,3 BUTYLENE GLYCOL VEGETAL | 5.0000 |
| PROPANEDIOL | 3.0000 |
| PENTYLENE GLYCOL VEG | 1.0000 |
| HYACROSS TL 200 (according to the invention) | 0.5000 |
| BASHYAL POWDER (according to the invention) | 0.3000 |

The facial plumper is a moisturizing and smoothing treatment for the face, ideal for application in the morning and/or evening. Applied to the skin of the face and/or neck, especially around wrinkles and fine lines, it provides an immediate smoothing effect, reducing roughness and smoothing skin micro-relief. The skin is visibly smoothed, clearer and younger.

### Lip Plumper

TABLE 10

| INGREDIENTS | % |
|---|---|
| TRI CAPRAT/CAPRYLAT GLYCEROL MB | 9.0000 |
| FAT | 30 |
| SENSIVA SC 50 | 0.3000 |
| PURIFIED WATER | QS 100 |
| HYACROSS TL 200 (according to the invention) | 0.2000 |
| OLIGO HA (according to the invention) | 0.3000 |
| LW GLYCEROL VEGETAL | 10.0000 |
| LW POLYGLYCERIN 500 | 5.0000 |
| LW PGL-S | 1.0000 |
| AQUAXYL | 3.0000 |
| D. PANTHENOL | 1.0000 |

The lip plumper is a moisturizing and plumping treatment for the lips. After application to the lips, fine lines are less visible, the lips appear fuller and plumper. The smoothing effect is immediate.

1. A cosmetic composition comprising, in a physiologically acceptable medium, the combination of at least one high molecular weight crosslinked hyaluronic acid having a molecular weight greater than or equal to 2000 kDa, or one of its salts, and a low molecular weight non-crosslinked hyaluronic acid having a molecular weight less than or equal to 300 kDa, or one of its salts.

2. The cosmetic composition according to claim 1, wherein said high molecular weight cross-linked hyaluronic acid has a molecular weight greater than or equal to 3000 kDa.

3. The cosmetic composition according to claim 1, wherein said low molecular weight non-cross-linked hyaluronic acid has a mean molecular weight less than or equal to 200 kDa.

4. The cosmetic composition according to claim 1, wherein said high molecular weight crosslinked hyaluronic acid has the INCI name: SODIUM HYALURONATE CROSSPOLYMER.

5. The cosmetic composition according to claim 1, wherein said low molecular weight non-cross-linked hyaluronic acid is chosen from products with INCI name:
SODIUM HYALURONATE, and
HYALURONIC ACID.

6. The cosmetic composition according to claim 1, wherein said high molecular weight cross-linked hyaluronic acid has a cross-linking of 0.001% to 5%.

7. The cosmetic composition according to claim 1, wherein said high molecular weight cross-linked hyaluronic acid and said low molecular weight non-cross-linked hyaluronic acid are each present in the composition in a content ranging from 0.001% to 10% by weight of raw material relative to the total weight of the composition.

8. The cosmetic composition according to claim 1, wherein it further comprises at least one cosmetic adjuvant chosen from the group consisting of antioxidant agents, emollient agents, moisturizing agents, anti-aging agents, fragrances, and mixtures thereof.

9. The cosmetic composition according to claim 1, wherein it comprises less than 5% filler.

10. The cosmetic composition according to claim 1, wherein it is in the form of a topical composition for the skin and/or lips.

11. A cosmetic process for caring for keratin materials, comprising the topical application to said keratin materials of at least one layer of the composition as defined in claim 1.

12. The cosmetic process according to claim 11, wherein it is intended to promote hydration of keratin materials and/or an immediate smoothing effect of keratin materials and/or to promote the firmness and/or elasticity and/or density and/or volume of keratin materials, and thus to prevent and/or reduce the signs of skin aging, in particular to prevent and/or reduce a loss of firmness and/or elasticity, and/or density and/or volume of the skin and/or lips, and/or the appearance of wrinkles.

13. The cosmetic process according to claim 12, wherein said composition is applied to healthy subjects presenting signs related to aging of the skin and/or lips.

14. (canceled)

15. The cosmetic composition according to claim 6, wherein said high molecular weight cross-linked hyaluronic acid has a cross-linking of 0.1% to 1%.

16. The cosmetic composition according to claim 7, wherein said high molecular weight cross-linked hyaluronic acid and said low molecular weight non-cross-linked hyaluronic acid are each present in the composition in a content ranging from 0.005% to 5% by weight of raw material relative to the total weight of the composition.

17. The cosmetic composition according to claim 7, wherein said high molecular weight cross-linked hyaluronic acid and said low molecular weight non-cross-linked hyaluronic acid are each present in the composition in a content ranging from 0.01% to 1% by weight of raw material relative to the total weight of the composition.

18. The cosmetic composition according to claim 9, wherein it comprises less than 2% filler.

19. The cosmetic composition according to claim 10, wherein it is in the form of a cream, oil-in-water or water-in-oil emulsion or multiple emulsion, suspension, gel, milk, lotion, serum, mask, or lip balm.

20. The cosmetic process according to claim 11, wherein the keratin materials are the skin and/or lips.

21. The cosmetic process according to claim 13, wherein the signs related to aging of the skin and/or lips are a loss of firmness and/or elasticity, and/or density and/or volume of the skin and/or lips, and/or wrinkles of the skin and/or on the contour of the lips and/or thin lips and/or lips presenting lines.
